(19) United States
(12) Patent Application Publication
(10) Pub. No.: US 2026/0043022 A1
(43) Pub. Date: Feb. 12, 2026

(54) Method of Modulating Production of Secondary Metabolites

(71) Applicant: Agency for Science, Technology and Research, Singapore (SG)

(72) Inventors: Dillon Wei Peng Tay, Singapore (SG); Yee Hwee Lim, Singapore (SG); Fong Tian Wong, Singapore (SG); Siew Bee Ng, Singapore (SG); Yoganathan Kanagasundaram, Singapore (SG)

(21) Appl. No.: 18/998,380

(22) PCT Filed: Jul. 14, 2023
(86) PCT No.: PCT/SG2023/050496

§ 371 Date: Jan. 24, 2025

(30) Foreign Application Priority Data
Jul. 27, 2022 (SG) 10202250605H

(51) Int. Cl. C12N 15/10

(52) U.S. Cl. CPC C12N 15/1082

(57) ABSTRACT

The invention relates to a method of modulating production of a metabolite in an actinomycetes bacterium comprising increasing the expression or activity of the Sporulation and Antibiotic Production Related gene A (SarA. SCO4069) polypeptide to modulate production of the metabolite in the actinomycetes bacterium. In one embodiment, the metabolite is valinomycin, daidzein, fluvirucin, demethyllydicamycin, or TPU-0037 analogues A. C and D.

### FIELD OF INVENTION

The invention relates generally to the field of microbiology. In particular, the specification teaches a method of modulating production of a metabolite in an actinomycetes bacterium.

### BACKGROUND

About 50% of current commercial drugs are derived from natural product (NP) or their mimics. A prolific producer of these bioactive complex compounds are Actinomycetes, among which are Streptomyces, which are important for the production of many clinically important antibiotics. With the advent of genomics technology and better whole genome sequencing data, it is now predicted that >80% of biosynthetic pathways within these bacteria are silent under laboratory conditions. This presents an opportunity for drug screening by activating these biosynthetic pathways. To do so, many have look towards genome editing or heterologous engineering. However, these approaches are challenging due to the requirements for specific genomic information and precise engineering and design.

Accordingly, it is generally desirable to overcome or ameliorate one or more of the above mentioned difficulties.

### SUMMARY

Disclosed herein is a method of modulating production of a metabolite in an actinomycetes bacterium, the method comprising increasing the expression or activity of the Sporulation and Antibiotic Production Related gene A (SarA) polypeptide to modulate production of the metabolite in the actinomycetes bacterium.

Disclosed herein is a method of screening for a metabolite produced by an actinomycetes bacterium, the method comprising a) increasing the expression or activity of the Sporulation and Antibiotic Production Related gene A (SarA) polypeptide in an actinomycetes bacterium to modulate production of the metabolite in the actinomycetes bacterium; and b) screening for production of the metabolite in the actinomycetes bacterium.

Disclosed herein is a method of preparing a drug compound library, the method comprising a) increasing the expression or activity of the Sporulation and Antibiotic Production Related gene A (SarA) polypeptide in an actinomycetes bacterium to modulate production of metabolites in the actinomycetes bacterium; and b) isolating metabolites from the actinomycetes bacterium to prepare the drug compound library.

Disclosed herein is an engineered actinomycetes bacterium comprising a Sporulation and Antibiotic Production Related gene A (SarA) gene operably linked to a promoter.

Disclosed herein is an actinomycetes bacterium engineered to overexpress the Sporulation and Antibiotic Production Related gene A (SarA) gene.

Disclosed herein is the use of an engineered actinomycetes bacterium as defined herein for producing a metabolite.

### BRIEF DESCRIPTION OF DRAWINGS

Embodiments of the present invention are hereafter described, by way of non-limiting example only, with reference to the accompanying drawings in which:

FIG. 1. SarA as global activator in Streptomyces. Metabolite maps were constructed by employing a dimension-reducing algorithm (t-distributed stochastic neighbour embedding, t-SNE) on compound fingerprints of mutants from 10 different Streptomyces strains to project it in 2D space. Metabolite signals are at least 10\hat{ }5 base peak abundance by liquid chromatography-mass spectrometry (LC-MS).

FIG. 2. Metabolite profiles of selected strains activated by SarA. (Only metabolite signals that are at least 10\hat{ }5 base peak abundance by liquid chromatography-mass spectrometry, LC-MS) (a) Comparison of new metabolites produced under different activators SarA, FAS, CRP and AdpA. Metabolites in the overlapped sections are produced in mutants with either activator incorporated. (b) Number of distinct metabolites produced by the 5 strains. Overall, 49 new metabolites were observed by SarA activation. Of the 32 metabolites observed in both native and SarA activated strains, 16 were upregulated by up to 18-fold. In these strains, 28 metabolites have disappeared from the mutants.

FIG. 3. LC-MS of fermentation extract from Streptomyces sp. A1532 in CA02LB media. Top: mass spectrum at 8.37 min. Mid: base peak chromatogram (BPC). Bottom: extracted ion chromatogram (EIC) for 1128.5+/−0.5 m/z corresponding to [M+NH4]+ for Valinomycin (boxed in red). EIC area=250.

FIG. 4. LC-MS of fermentation extract from Streptomyces sp. A1532 SarA mutant A100280 in CA02LB media. Top: mass spectrum at 8.37 min. Mid: base peak chromatogram (BPC). Bottom: extracted ion chromatogram (EIC) for 1128.5+/−0.5 m/z corresponding to [M+NH4]+ for Valinomycin (boxed in red). EIC area=14,722 (59-fold increase from wild type strain).

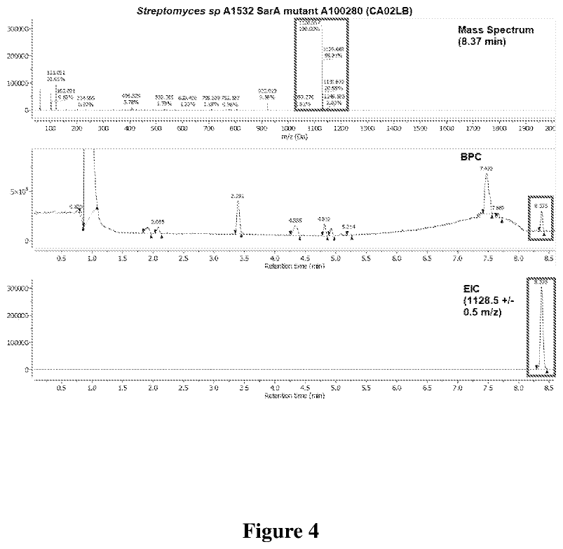

FIG. 5. LC-MS of fermentation extract from Streptomyces sp. A34001 in CA02LB media. Top: mass spectrum at 3.20 min. Mid: base peak chromatogram (BPC). Bottom: extracted ion chromatogram (EIC) for 255+/−0.5 m/z corresponding to [M+H]+ for Daidzein (boxed in red). EIC area=466.

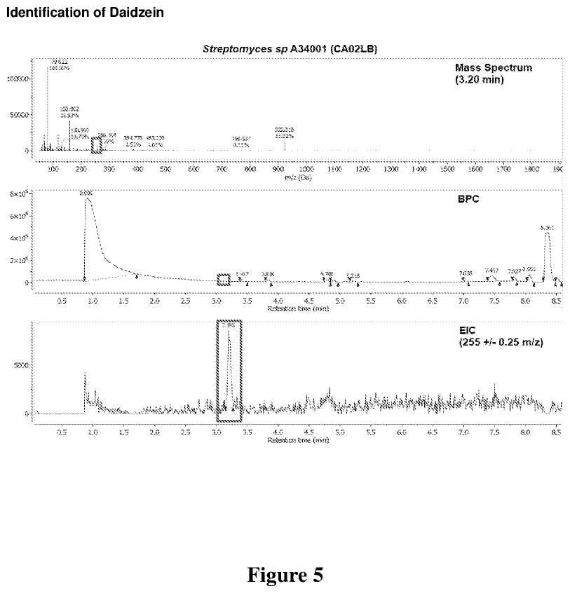

FIG. 6. LC-MS of fermentation extract from Streptomyces sp. A34001 SarA mutant A100270 in CA02LB media. Top: mass spectrum at 3.39 min. Mid: base peak chromatogram (BPC). Bottom: extracted ion chromatogram (EIC) for 255+/−0.5 m/z corresponding to [M+H]+ for Daidzein (boxed in red). EIC area=19,361 (42-fold increase from wild type strain).

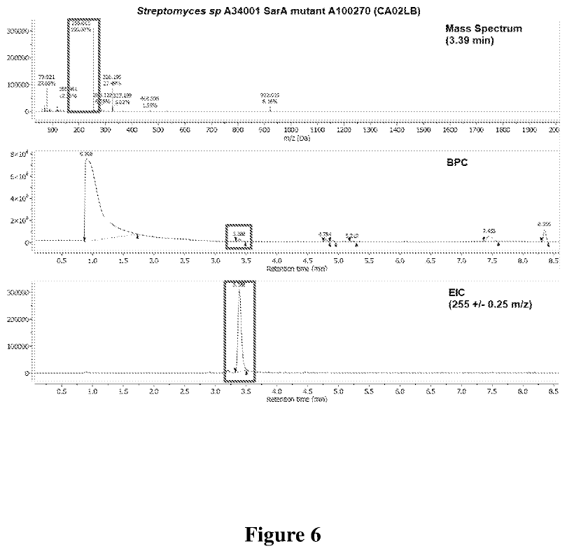

FIG. 7 Upregulation of TPU-0037 analogs in Streptomyces sp A80510 and its edited strains. Left: fold change comparisons of normalized yields averaged from 3 mutants, error bars given for 1 standard deviation. Right: chemical structures of TPU-0037 A, C and D analogs.

### DETAILED DESCRIPTION

The present specification teaches a method of modulating production of metabolites in an actinomycetes bacterium. Disclosed herein is a method of modulating production of a metabolite in an actinomycetes bacterium, the method comprising increasing the expression or activity of the Sporulation and Antibiotic Production Related gene A (SarA) polypeptide to modulate production of the metabolite in the actinomycetes bacterium.

Without being bound by theory, it was found that increasing the expression or activity of the SarA polypeptide alone was sufficient to modulate production of metabolites in actinomycetes bacterium. The method may increase or decrease the production of the metabolite in the actinomycetes bacterium. For example, the method may increase the production of the metabolite by 1, 2, 3, 4, 5, 6, 7, 8, 9, 10, 11, 12, 13, 14, 15, 16, 17, 18, 19, 20 or more fold as compared to a reference.

The method as defined herein may comprise increasing the expression or activity of the Sporulation and Antibiotic Production Related gene A (SarA) polypeptide. The method may increase the expression or activity of the SarA polypeptide by 1, 2, 3, 4, 5, 6, 7, 8, 9, 10, 11, 12, 13, 14, 15, 16, 17, 18, 19, 20 or more fold as compared to a reference. The reference may refer, for example, refer to an actinomycetes bacterium that has not been exposed to the step of increasing the expression or activity of the SarA polypeptide. Alternatively, it may refer to a predetermined level of SarA polypeptide.

The method may comprise genetically modifying the actinomycetes bacterium to have increased expression or activity of the SarA polypeptide. The actinomycetes bacterium may have increased expression or activity of the SarA polypeptide as compared to a reference.

The term “genetically engineer” or “genetically modify” as used herein refers to the modification of the actinomycetes bacteria's genetic make-up using molecular biological methods. The modification of the genetic make-up may include the transfer of genes within and/or across species boundaries, inserting, deleting, replacing and/or modifying nucleotides, triplets, genes, open reading frames, promoters, enhancers, terminators and other nucleotide sequences mediating and/or controlling gene expression. The modification of the genetic make-up aims to generate a genetically modified bacterium cell possessing particular desired properties (e.g. increased SarA polypeptide expression). Genetically engineered actinomycetes cells can contain one or more genes that are not present in the native (not genetically engineered) form of the cell. Techniques for introducing exogenous nucleic acid molecules and/or inserting exogenous nucleic acid molecules (recombinant, heterologous) are well known to the skilled artisan. Genetically engineered actinomycetes cells can contain one or more genes that are present in the native form of the cell, wherein said genes are modified and re-introduced into the cell by artificial means. The term “genetically engineered” also encompasses actinomycetes cells that contain a nucleic acid molecule being endogenous to the cell, and that has been modified without removing the nucleic acid molecule from the cell. Such modifications include those obtained by gene replacement, site-specific mutations, and related techniques. Gene integration and/or gene inactivation by disruption or deletion can be achieved by homologous recombination. The term “genetically engineer” or “genetically modify” may refer to the transient engineering or modification of the bacterial genome.

In one embodiment, there is provided a method of modulating production of a metabolite in an actinomycetes bacterium, the method comprising increasing the expression or activity of the Sporulation and Antibiotic Production Related gene A (SarA) polypeptide by genetic modification of the of the actinomycetes bacterium to modulate production of the metabolite in the actinomycetes bacterium.

In one embodiment, the actinomycetes bacterium is genetically modified using site-specific recombination. For example, the the actinomycetes bacterium is genetically modified using an integrase/att system from bacteriophage lambda.

It is known in the art that it is possible to use a site-specific nuclease to make a DNA break in the genome of a living cell, and that such a DNA break can result in permanent modification of the genome via mutagenic non-homologous end joining (NHEJ) repair or via homologous recombination with an exogenous DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. The use of nucleases to induce a double-strand break in a target locus is known to stimulate homologous direct repair (HDR), particularly of transgenic DNA sequences flanked by sequences that are homologous to the genomic target. In this manner, exogenous nucleic acid sequences can be inserted into a target locus. Such exogenous nucleic acids can encode any sequence of interest, such as a SAR encoding nucleic acid. A variety of site-specific nucleases can be used. For example, the nuclease may be a recombinant meganuclease, a CRISPR nuclease, a zinc finger nuclease (ZFN) or a transcription activator-like effector nuclease (TALENs).

In various embodiments, a CRISPR (Clustered Regularly Interspaced Short Palindromic Repeats)/Cas (CRISPR Associated) nuclease system is engineered to bind to, and to introduce single-stranded nicks or double-strand breaks (DSBs) in, one or more target sites. The CRISPR/Cas nuclease system is a recently engineered nuclease system based on a bacterial system that can be used for mammalian genome engineering. Engineered endonucleases based on the CRISPR/Cas9 system are also known in the art. A CRISPR endonuclease comprises two components: (1) a caspase effector nuclease, typically microbial Cas9; and (2) a short “guide RNA” comprising a nucleotide targeting sequence that directs the nuclease to a location of interest in the genome.

The term “CRISPR” refers to a caspase-based endonuclease comprising a caspase, such as Cas9, and a guide RNA that directs DNA cleavage of the caspase by hybridizing to a recognition site in the genomic DNA.

In some embodiments the CRISPR/Cas system is CRISPR/Cas9. In some embodiments the CRISPR/Cas system comprises at least one nucleic acid encoding a CRISPR nuclease and at least one nuclease acid encoding a guide RNA. The two nucleic acids may be introduced separately, e.g., in different vectors or separate mRNAs, or together, e.g., in a polycistronic construct separated by a viral self-cleaving peptide or an IRES element.

In some embodiments, the CRISPR/Cas system comprises an exogenous sequence for integrating into the genome via homologous recombination following cleavage by the nuclease. The design of exogenous sequences for replacing a native sequence in CRISPR/Cas gene editing is well known in the art.

The method may comprise introducing an expression construct comprising a heterologous SarA gene that is operably linked to a promoter to the actinomycetes bacterium. The heterologous SarA gene may be SCO 4069 or its homologues. In one embodiment, the method comprises overexpressing SarA gene in the actinomycetes bacterium. The expression construct may be introduced transiently into the actinomycetes bacterium using techniques that are well known in the art (such as via transformation). Alternatively, the expression construct may be stably integrated into the genome of the actinomycetes bacterium.

Alternatively, the method may comprise increasing endogenous expression or activity of the SarA gene. This may be done so by swapping out the promoter of the endogenous SarA genes. For example, the endogenous SarA gene may be genetically modified to be placed under the control of a strong constitutive promoter (such as kasOp*).

Other methods for increasing the endogenous expression or activity of the SarA gene are also contemplated. For example, one may contact the actinomycetes bacterium with one or more exogenous agents (such as a small molecule, peptide or nucleic acid molecule) that inhibit degradation of SarA protein or mRNA.

The terms “polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only.

As used herein, the term “overexpression” and “upregulation” or “increasing expression” of a polypeptide refers to the expression of a nucleic acid encoding a polypeptide (e.g., a gene) in a modified cell at higher levels (therefore producing an increased amount of the polypeptide encoded by the gene) as compared to a “wild type” cell (e.g., a substantially equivalent cell that is not modified in the manner of the modified cell) under substantially similar conditions. Thus, to overexpress or increase expression of SarA polypeptide refers to increasing or inducing the production of the SarA polypeptide, which may be done by a variety of approaches, such as, but not limited to: increasing the transcription of the SarA gene (such as by placing the endogenous SarA gene under the control of a strong promoter or introducing a heterologous SarA gene into the cell), or by increasing the translation of the SarA gene, or a combination of these and/or other approaches.

The term “gene” as used herein refers to any and all discrete coding regions of the cell's genome, as well as associated non-coding and regulatory regions. The term is intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The term “encoding” or “encodes” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e. rRNA, tRNA and mRNA) or a defined sequence of amino acids and the biological properties resulting therefrom. Thus, a gene encodes a protein if transcription of a gene and translation of mRNA corresponding to that gene produces the protein in a cell or other biological system. Both the coding strand and the non-coding strand, used as the template for transcription of a gene or cDNA, can be referred to as encoding the protein or other product of that gene or cDNA.

The actinomycetes bacterium may be a Streptomyces bacterium.

In one embodiment, the Streptomyces is selected from Streptomyces sp. A34001, Streptomyces sp. A1532, Streptomyces sp. A33995, Streptomyces sp. A5252, Streptomyces sp. A1137, Streptomyces sp. A80510, Streptomyces sp. A2056, Streptomyces sp. A1123, Streptomyces sp. A53691, Streptomyces sp. T1236 or Streptomyces fulvissimus. The Streptomyces may be Streptomyces sp. A1532 or Streptomyces sp. A33995. The Streptomyces may be Streptomyces sp. A1532. The Streptomyces may be Streptomyces sp. A34001.

In one embodiment, the actinomycetes bacterium is selected from Streptomyces niveiscabiei, Micromonospora maritima, Streptomyces lunaelactis, Thermoactinomyces vulgaris, Micromonospora maritima, Streptomyces ardesiacus, Thermoactinomyces vulgaris, Micromonospora aurantiaca and Streptomyces ardesiacus.

In one embodiment, the metabolite is a secondary metabolite. Secondary metabolites typically are organic compounds that are not directly involved in the normal growth, development, or reproduction of an organism. Unlike primary metabolites, absence of secondary metabolites does not result in immediate death, but rather in long-term impairment of the organism's survivability, fecundity, or aesthetics, or perhaps in no significant change at all. In one embodiment, the secondary metabolite is an antibiotic, an antibiotic resistance inhibitor, an anti-cancer compound, an enzyme inhibitor, an antifungal, an antihelminthic, an immunostimulant, an immunosuppressant, an insecticide or a herbicide. In one embodiment, the secondary metabolite has anti-microbial activity. A class of antibiotic resistance inhibitors are compounds that increase the sensitivity to an antibiotic of a bacterium that is resistant to the antibiotic under physiological conditions. An example of such compound is clavulanic acid. Such products can, for instance, be evaluated by growing the product producing micro-organism in the presence of the resistant micro-organism. In one embodiment, the secondary metabolite is an alkaloid or an antibiotic. Preferred antibiotics are antibiotics of the following groups: actinorhodin, valinomycin, fluvirucin, demethyllydicamycin, actinoaminoglycosides (e.g., kanamycin, neomycin, streptomycin), ansamycins, carbapenems, cephalosporins, glycopeptides (e.g., vancomycin, teichoplanin, daptomycin), lantibiotics (e.g., actagardin, mersacydin, nisin), lincosamides (e.g., clindamycin, lincomycin), macrolides (e.g., azithromycin, erythromycin, spectinomycin), penicillins (ampicillin, methicillin, penicillin G), polypeptides (e.g., bactitracin), quinolones (e.g., cirpofloxacin, nalidixic acid), rifamycins (e.g., rifampicin), sulfonamides (e.g., trimethoprim), tetracyclins, tuberactinomycins (e.g., capreomycin, viomycin), and chloramphenicol. In one embodiment, the metabolite is an anti-cancer compound. In one embodiment, the metabolite is daidzein. In one embodiment, the metabolite is valinomycin. daidzein, fluvirucin or demethyllydicamycin. In one embodiment, the metabolite is valinomycin. In one embodiment, the metabolite is fluvirucin. The fluvirucin may be fluvirucin B1 or fluvirucin B3. In one embodiment, the metabolite is demethyllydicamycin.

In one embodiment, the metabolite is TPU-0037 or an analogue thereof. In one embodiment, the metabolite is TPU-0037 analogues A, C or D. In one embodiment, the metabolite is a compound having the formula of:

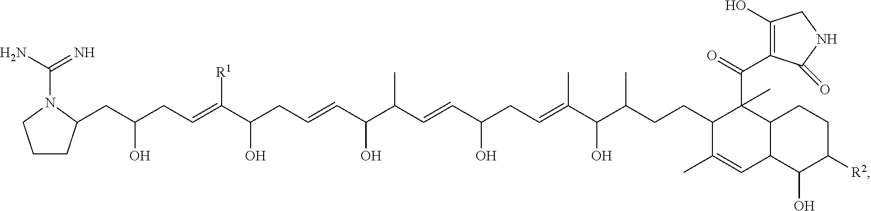

wherein R¹ is H or Me and wherein R² is H or OH.

Disclosed herein is a method of screening for a metabolite produced by an actinomycetes bacterium, the method comprising a) increasing the expression or activity of the Sporulation and Antibiotic Production Related gene A (SarA) polypeptide in an actinomycetes bacterium to modulate production of the metabolite in the actinomycetes bacterium; and b) screening for production of the metabolite in the actinomycetes bacterium.

Provided herein is also a method of preparing a drug compound library. The method may include increasing the expression or activity of the Sporulation and Antibiotic Production Related gene A (SarA) polypeptide in an actinomycetes bacterium to modulate production of metabolites in the actinomycetes bacterium. The method may include isolating metabolites that are produced by the actinomycetes bacterium to prepare a drug compound library.

Disclosed herein is a method of preparing a drug compound library, the method comprising a) increasing the expression or activity of the Sporulation and Antibiotic Production Related gene A (SarA) polypeptide in an actinomycetes bacterium to modulate production of metabolites in the actinomycetes bacterium; and b) isolating metabolites from the actinomycetes bacterium to prepare the drug compound library. The method may include screening the drug compound library to identify compounds with desirable activities.

Disclosed herein is a construct comprising an isolated polynucleotide as defined herein.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest. Within certain embodiments of the invention, the construct may be contained within a vector. In addition to the components of the construct, the vector may include, for example, one or more selectable markers, one or more origins of replication, such as prokaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors.

Disclosed herein is an expression construct comprising a Sporulation and Antibiotic Production Related gene A (SarA) gene operably linked to a promoter.

An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

The isolated polynucleotide may be operably linked to a control element.

By “control element” or “control sequence” is meant nucleic acid sequences (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site.

Disclosed herein is a vector comprising a construct as defined herein.

By the term “vector”, as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of a nucleic acid that encodes a polypeptide of the present invention to the patient, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a lentiviral vector, a recombinant adenovirus, a recombinant retrovirus, a recombinant adeno-associated virus, a recombinant avian pox virus, and the like. Examples of non-viral vectors include, but are not limited to, liposomes, polyamine derivatives of DNA, and the like.

Provided herein is an actinomycetes bacterium engineered to overexpress Sporulation and Antibiotic Production Related gene A (SarA) gene. The actinomycetes bacterium may be further engineered to overexpress other regulators such as CRP, FAS and/or AdpA.

Disclosed herein is an engineered actinomycetes bacterium comprising a Sporulation and Antibiotic Production Related gene A (SarA) gene operably linked to a promoter.

In one embodiment, the promoter is a strong constitutive promoter, such as kasOp* promoter.

Disclosed herein is the use of an engineered actinomycetes bacterium as defined herein for producing a metabolite.

Also provided herein is the use of an engineered actinomycetes bacterium as defined herein for producing a metabolite library.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Methods

Strains Used in this Study

Strains were obtained from Natural Organism Library (NOL, [8]). NOL is a rich biodiverse resource comprising of more than 100,000 natural microorganisms isolated from diverse habitats.

### Strains:

A34001
A1532
A33995
A5252
A1137
A80510
A2056
A1123
A53691
T1236

### Integration Cassette for SarA

Overexpression SarA cassette consisting of kasOp*-SCO4069 (accession code: WP_011029418). kasOp* is a strong constitutive promoter [6]. The integration plasmid was derived by inserting the overexpression cassette into pSET152 [7]. The completed plasmid was conjugated into various native Streptomyces strains from the natural organism library collection (SIFBI, NOL). Integration is mediated by attP site of the Streptomyces phage ΦC31. Genetically integrated mutants were screened and sequenced and then applied for fermentation.

### Fermentation and Analysis

Wild type Streptomyces and edited mutants were cultured in 5 mL SV2 media for 3-5 days. Saturated seed cultures were diluted into fresh fermentation media: CA02LB, CA07LB, CA08LB, CA09LB and CA10LB in a 1:20 volume ratio and fermented with 200 rpm shaking at 30° C. After 9 days, the cultures were pelleted then the separated biomass and supernatant were lyophilized. The dried samples were extracted by methanol then filter through filter paper (Whatman Grade 4) and filtrates were reconstituted for analysis.

The extracts were analysed on an Agilent 1290 Infinity LC System coupled to an Agilent 6540 accurate-mass quadrupole time-of-flight (QTOF) mass spectrometer. 5 μL of extract was injected onto a Waters Acquity UPLC BEH C18 column, 2.1×50 mm, 1.7 μm. Mobile phases were water (A) and acetonitrile (B), both with 0.1% formic acid. The analysis was performed at flow rate of 0.5 mL/min, under gradient elution of 2% B to 100% B in 8 min. Both MS and MS/MS data were acquired in positive electrospray ionization (ESI) mode. The typical QTOF operating parameters were as follows: sheath gas nitrogen, 12 L/min at 325° C.; drying gas nitrogen flow, 12 L/min at 350° C.; nebulizer pressure, 50 psi; nozzle voltage, 1.5 kV; capillary voltage, 4 kV. Lock masses in positive ion mode: purine ion at m/z 121.0509 and HP-0921 ion at m/z 922.0098.

### Data Analysis

Compounds were detected based on peaks identified in base peak chromatograms (BPC) from LC-MS data. ESI (+ve) mass spectral data at the apex of detected peaks were sampled and corrected for background noise by deducting the nearest baseline mass spectra. Detected peaks were then characterized with 4 major parameters: (1) base peak m/z, (2) retention time, (3) molecular ion peak and (4) number of m/z peaks exceeding 50,000 abundance. Peaks were considered to be identical if they fulfilled 2 criteria: (1) base peak m/z within 0.02 m/z and (2) retention time within 0.2 min.

### Example 1

### Results

50% of current commercial drugs have been derived from natural product (NP) or their mimics. A prolific producer of these bioactive complex compounds are Actinomycetes and among which are Streptomycetes. Although there has been a decrease in drug discovery rates, with the advent of genomics technology and better whole genome sequencing data of these bacteria, it is now predicted that >80% of biosynthetic pathways within these bacteria are silent under lab conditions. To activate these pathways, genome editing or heterologous engineering have been used to restart the pathways. However, these required genomic information and precise engineering and design. To circumvent this, a general and highly efficient workflow to activate biosynthetic production is required.

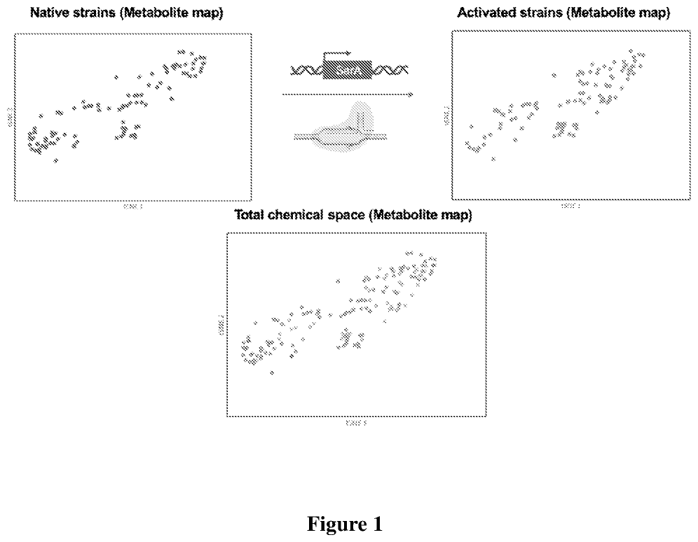

A high throughput general workflow which enables high efficiencies in activating production of new and previously silent bioactives in Streptomyces, was established. This workflow integrates overexpression cassettes into genomes of Streptomyces strains which then enable activation or increased production of secondary metabolites (FIG. 1). Activators that regulate natural products production on a global scale had demonstrated prior high rates of activation. These include known regulators CRP [1], FAS [2] and AdpA [3]. For example, CRP overexpression upregulated expression of 6 NPs out of 22 clusters in Streptomyces coelicolor genome. However, the encoded genetic space of microbial strains suggest that a vast majority of the chemical space remains to be discovered.

To tap into potentially new chemical space, the inventors integrated SarA into at least 7 Streptomyces strains and showed that it is an effective global activator to upregulate the production of new secondary metabolites in Streptomyces.

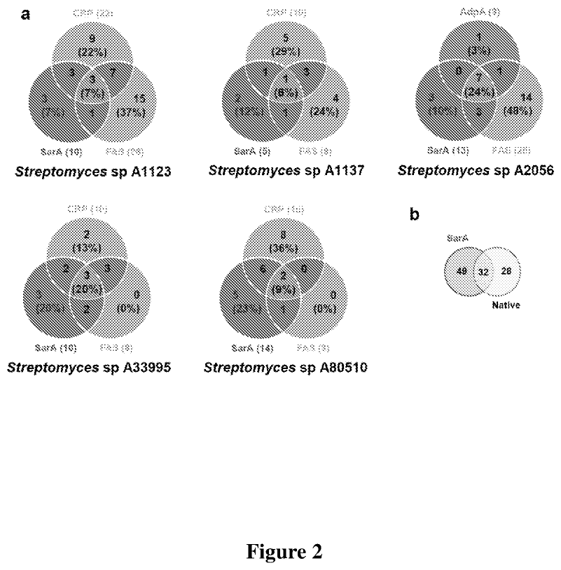

The inventors have designed and tested an overexpression integration cassette for SarA and have demonstrated that SarA is able to function as a global activator of secondary metabolites in Streptomyces (FIG. 2).

Amongst 5 strains, Streptomyces sp. A1123, A1137, A2056, A33995, A80510, generation of 49 new metabolites in the SarA activated strains was observed. These new metabolites are not found in the native strains across all five media. Of new metabolites from global regulators (FIG. 2a), 16 metabolites (2-5 metabolites from each of the analyzed strain) were only produced under SarA activation conditions.

Moreover, SarA activation also resulted in upregulation of selected native metabolites. Amongst the 5 strains, there was upregulated production of 54 native strains produced metabolites in the SarA activated strains. (Note: only metabolites with MS signals (total/extracted ion chromatogram)>105 base peak abundance are considered here).

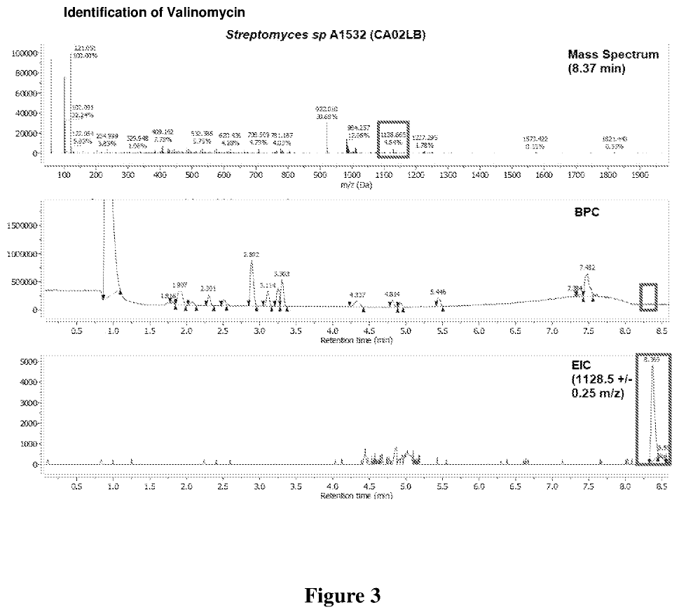

Though most of these metabolites have not been structurally elucidated, they are likely to be mostly complex natural products. For examples, Valinomycin and Daidzein are two known natural products identified in two of the studied strains. Valinomycin in Streptomyces sp A1532 and Daidzein in Streptomyces sp A34001 were significantly upregulated in their respective SarA overexpression mutants (FIG. 3, 6). These natural products were previously observed minimally in the wild type strains under lab conditions (FIG. 3, 5).

### Example 2

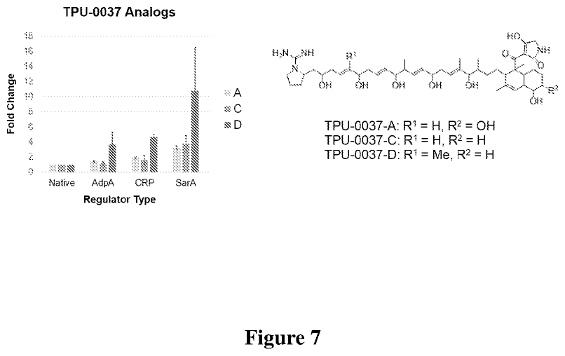

In Streptomyces sp. A80510 mutants, introducing activators resulted in up to 15-fold increased production of TPU-0037 analogues A, C and D versus the native strain with SarA exhibiting the greatest upregulation (FIG. 7). TPU-0037 analogues are known to possess bioactivity against Staphylococcus aureus.

### REFERENCES

1. Gao, Chan, et al. “Crp is a global regulator of antibiotic production in Streptomyces.” MBio 3.6 (2012): e00407-12.
2. Wang, Weishan, et al. “Harnessing the intracellular triacylglycerols for titer improvement of polyketides in Streptomyces.” Nature biotechnology 38.1 (2020): 76-83.
3. Lee H N, Kim J S, Kim P, Lee H S, Kim E S. Repression of antibiotic downregulator WblA by AdpA in Streptomyces coelicolor. Appl. Environ. Microbiol. 2013 Jul. 1; 79(13):4159-63.
4. Ou X, Zhang B, Zhang L, Dong K, Liu C, Zhao G, Ding X. 2008. SarA influences the sporulation and secondary metabolism in Streptomyces coelicolor M145. Acta Biochim Biophys Sin (Shanghai) 40:877-882. http://dx.doi.org/10.1093/abbs/40.10.877.
5. Xu Z, Li Y, Wang Y, Deng Z, Tao M. Genome-Wide Mutagenesis Links Multiple Metabolic Pathways with Actinorhodin Production in Streptomyces coelicolor. Appl. Environ. Microbiol. 2019 Apr. 1; 85(7): e03005-18.
6. Wang, Weishan, et al. “An engineered strong promoter for streptomycetes.” Applied and environmental microbiology 79.14 (2013): 4484-4492.
7. Bierman, M., et al. “Plasmid cloning vectors for the conjugal transfer of DNA from Escherichia coli to Streptomyces spp.” Gene 116.1 (1992): 43-49.
8. Ng, Siew Bee, et al. “The 160K natural organism library, a unique resource for natural products research.” Nature biotechnology 36.7 (2018): 570-573.

1. A method of modulating production of a metabolite in an actinomycetes bacterium, the method comprising increasing the expression or activity of the Sporulation and Antibiotic Production Related gene A (SarA) polypeptide to modulate production of the metabolite in the actinomycetes bacterium.

2. The method of claim 1, wherein the method increases or decreases the production of the metabolite in the actinomycetes bacterium.

3. The method of claim 1, wherein the metabolite is a secondary metabolite.

4. The method of claim 1, wherein the metabolite is valinomycin, daidzein, fluvirucin or demethyllydicamycin.

5. The method of claim 1, wherein the metabolite is a compound having the formula of: 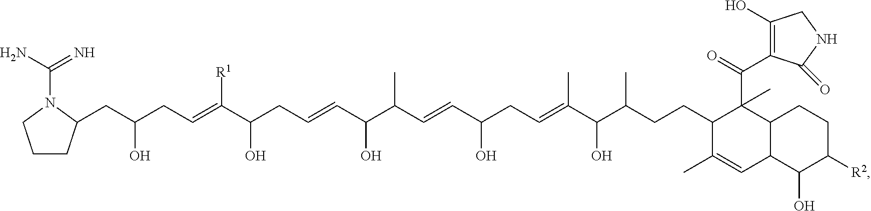
wherein R¹ is H or Me and wherein R² is H or OH.

6. The method of claim 1, wherein the actinomycetes bacterium is a Streptomyces bacterium.

7. The method of claim 1, wherein the method comprises genetically modifying the actinomycetes bacterium to have increased expression or activity of the SarA polypeptide.

8. The method of claim 1, wherein the method comprises introducing an expression construct comprising a heterologous SarA gene that is operably linked to a promoter to the actinomycetes bacterium.

9. The method of claim 1, wherein the method comprises culturing the actinomycetes bacterium so as to modulate the production of the metabolite in the actinomycetes bacterium.

10. A method of screening for a metabolite produced by an actinomycetes bacterium, the method comprising a) increasing the expression or activity of the Sporulation and Antibiotic Production Related gene A (SarA) polypeptide in an actinomycetes bacterium to modulate production of the metabolite in the actinomycetes bacterium; and b) screening for production of the metabolite in the actinomycetes bacterium.

11. The method of claim 10, wherein the method comprises introducing an expression construct comprising a heterologous SarA gene that is operably linked to a promoter.

12. A method of preparing a drug compound library, the method comprising a) increasing the expression or activity of the Sporulation and Antibiotic Production Related gene A (SarA) polypeptide in an actinomycetes bacterium to modulate production of metabolites in the actinomycetes bacterium; and b) isolating metabolites from the actinomycetes bacterium to prepare the drug compound library.

13-15. (canceled)
